(19) United States
(12) Patent Application Publication
(10) Pub. No.: US 2019/0275169 A1
(43) Pub. Date: Sep. 12, 2019

(54) TARGETED IMMUNOSUPPRESSION FOR OPTOGENETIC INTERVENTION

(71) Applicant: Circuit Therapeutics, Inc., Mountain View, CA (US)

(72) Inventors: Christopher L. Towne, San Francisco, CA (US); Claire Discenza, Redwood City, CA (US); Sean McCormack, Santa Maria, CA (US)

(73) Assignee: Circuit Therapeutics, Inc., Mountain View, CA (US)

(21) Appl. No.: 16/296,063

(22) Filed: Mar. 7, 2019

Related U.S. Application Data
(60) Provisional application No. 62/639,961, filed on Mar. 7, 2018.

(51) Int. Cl. A61K 48/00; A61K 31/436; C12N 15/86; C07K 14/705

(52) U.S. Cl. CPC A61K 48/0058; A61K 31/436; C07K 14/705; C12N 15/86; A61K 48/0083

(57) ABSTRACT

One embodiment is directed to a method for treating the nervous system of a patient, comprising: determining a desired nervous system functional modulation to be facilitated by optogenetic intervention; selecting targeted neuroanatomy for achieving the desired functional outcome with optogenetic intervention; and delivering an effective amount of polynucleotide comprising a light-responsive opsin protein which is expressed in neurons of the targeted neuroanatomy, and delivering an effective amount of immunosuppressant.

### RELATED APPLICATION DATA

The present application claims priority to U.S. Provisional Application Ser. No. 62/639,961, filed Mar. 7, 2018. The foregoing application is hereby incorporated by reference into the present application in its entirety.

### FIELD OF THE INVENTION

The present invention relates generally to systems and methods for treatment of pathological neurological conditions utilizing optogenetic therapies.

### BACKGROUND

The emerging field of optogenetics provides a means to control cellular function through the selective delivery of light to a cell that has been transformed with genetic information encoding an opsin, or a protein that is light-sensitive. Opsins are a family of proteins that perform ion transport when exposed to light. The opsin is transduced into the cell, commonly using viral vectors or any other method utilized by gene transfer, and is therefore expressed on the cell's membrane. When light is applied, the ion transport afforded by the opsin changes the function of the cell in a light-dependent, or light-responsive, manner. Various aspects of optogenetic fundamentals are described by Karl Deisseroth, for example, in Optogenetics, Nature Methods, 8: 26-29 (2011), and in U.S. patent application Ser. Nos. 14/444,722, 14/737,445, 14/737,446, 15/428,097, and 15/911,070; each of these six references is incorporated by reference in its entirety herein.

A wide variety of opsins may be utilized to either excite or inhibit the production of action potentials within the neurons of a particular neurological circuit with the delivery of light. The result is dependent on the identity and direction of the ion transport provided by the opsin. In particular, if the ion is positively charged and the movement is into the cell or if the ion is negatively charged and the movement is out of the cell, increased transport results in a stimulation of the cell expressing the opsin (e.g. an increased chance of an action potential, or depolarization). This is generally known as “stimulation.” Further, if the ion is negatively charged and the movement is into the cell or the ion is positively charged and the movement is out of the cell, increased transport results in an inhibition of the cell expressing the opsin (e.g. a decreased chance of an action potential, or hyperpolarization). This is generally known as “inhibition.”

Utilizing the very selective and controllable aspects of inhibitory and stimulatory opsins, various interventions may be conducted, as well as experiments to better understand the precise function of various circuits of the neuroanatomy.

One of the challenges with introduction of exogenous proteins, such as via viral vector transport, into a live animal is the interplay between such genetic material and transport means with the immune systems of the target animals. In our experiments, we have found certain circumstances wherein it is desirable to modulate aspects of the immune system, as specifically as possible, to extend the observable functionality of opsin proteins. To address these challenges, we have developed certain treatment configurations which we have found to significantly assist in providing targeted immunosuppression for light responsive exogenous opsin proteins introduced into various species. Such configurations are described herein.

### SUMMARY

One embodiment is directed to a method for treating the nervous system of a patient, comprising: determining a desired nervous system functional modulation to be facilitated by optogenetic intervention; selecting targeted neuroanatomy for achieving the desired functional outcome with optogenetic intervention; and delivering an effective amount of polynucleotide comprising a light-responsive opsin protein which is expressed in neurons of the targeted neuroanatomy, and delivering an effective amount of immunosuppressant.

### BRIEF DESCRIPTION OF THE FIGURES

FIG. 1 illustrates a table of various drug combinations that are known to modulate the immune system, and specific schedules and dosages utilized in the subject experiments.

FIG. 2A illustrates a plot showing the proportion of experimental animals still exhibiting light-responsiveness versus time after initial viral genetic material introduction for a cyclosporine group versus a saline group.

FIG. 2B illustrates a plot showing the proportion of experimental animals still exhibiting light-responsiveness versus time after initial viral genetic material introduction for a cyclosporine+mycophenolate mofetil group versus a saline group.

FIG. 2C illustrates a plot showing the proportion of experimental animals still exhibiting light-responsiveness versus time after initial viral genetic material introduction for a dexamethasone group (administered before viral injection and also afterwards) versus a saline group.

FIG. 2D illustrates a plot showing the proportion of experimental animals still exhibiting light-responsiveness versus time after initial viral genetic material introduction for a dexamethasone group (at time of viral injection and also afterwards) versus a saline group.

FIG. 2E illustrates a plot showing the proportion of experimental animals still exhibiting light-responsiveness versus time after initial viral genetic material introduction for a low dosage level rapamycin group versus a saline group.

FIG. 2F illustrates a plot showing the proportion of experimental animals still exhibiting light-responsiveness versus time after initial viral genetic material introduction for a medium dosage level rapamycin group versus a saline group.

FIG. 2G illustrates a plot showing the proportion of experimental animals still exhibiting light-responsiveness versus time after initial viral genetic material introduction for a high dosage level rapamycin group versus a saline group.

FIG. 2H illustrates a plot showing the proportion of experimental animals still exhibiting light-responsiveness versus time after initial viral genetic material introduction for a rapamycin+FK506+dexamethasone group versus a saline group.

FIG. 2I illustrates a plot showing the proportion of experimental animals still exhibiting light-responsiveness versus time after initial viral genetic material introduction for a metformin group versus a saline group.

FIG. 3 illustrates a plot showing the proportion of experimental animals still exhibiting light-responsiveness versus time after initial viral genetic material introduction for various time-domain exposure rapamycin groups versus a saline group.

FIG. 4A illustrates histologic graphics depicting IC++opsin expression configurations (after direct dorsal root ganglia injection) at 30-days post injection, with and without (saline) rapamycin.

FIG. 4B numerically/graphically illustrates the proportion of experimental animals still exhibiting light-responsiveness at 30 days for two different rapamycin dosage groups versus a control (vehicle) group.

FIG. 4C numerically/graphically illustrates the proportion of experimental animals still exhibiting light-responsiveness at 100 days for two different rapamycin dosage groups versus a control (vehicle) group.

FIG. 5 illustrates that a rapamycin targeted immunosuppression configuration also extends in time the proportion of experimental animals still exhibiting light-responsiveness for motor neuron interventions.

FIG. 6 illustrates one embodiment of a method configuration wherein an immunosuppressant may be utilized in an optogenetic therapeutic intervention.

### DETAILED DESCRIPTION

As noted above, certain circumstances have been presented wherein it is desirable to modulate aspects of the immune system, as specifically as possible, to extend the observable functionality of opsin proteins from a time-after-introduction perspective. Rapamycin, also known as sirolimus, is a macrolide compound initially isolated in 1972 that has been utilized in various interventional applications, such as in the coating of cardiovascular stent implants to prevent endothelial cell proliferation which may be associated with re-stenosis of targeted vessels. In the interest of creating a targeted immunosuppression paradigm for optogenetic therapy, we hypothesized, conducted experiments, and have confirmed that in certain configurations it is effective in prolonging the observable light-responsiveness of opsin-modified tissues in various species. We believe that this is related to the up-regulation of T-regulator cells and down-regulation of T-effector cells, which provides higher immunosuppressant specificity and theoretical longevity as a treatment paradigm, as compared with other more systemic immunosuppressant drugs, such as cyclosporine or mycophenolate mofetil, which are believed to regulate all immune cells downward.

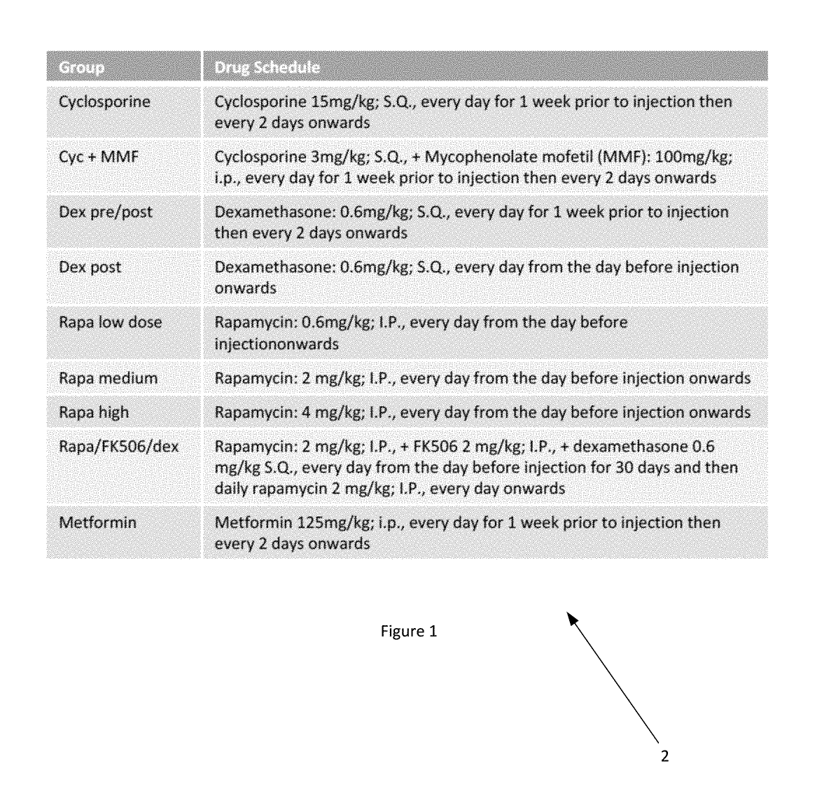

Referring to FIG. 1, various drug groupings are listed (2) which have been known to modulate the immune system, including cyclosporine (“Cyc”), cyclosporine+mycophenolate mofetil (“MMF”), dexamethasone, rapamycin in various dosage configurations, rapamycin+dexamethasone+FK506 (also known as “tacrolimus”; an immunosuppressive drug used mainly after organ transplant to lower rejection risk), and metformin (a medication utilized in treating diabetes and polycystic ovary syndrome which also has been inculcated in immune suppression). Each of these listed drugs has previously been approved by the U.S. F.D.A. for different indications, and the depicted drug schedules associated with each grouping feature dosing and scheduling information that previously has been published in different treatment paradigms, such as for cardiovascular or transplant rejection scenarios.

Thus, referring again to the table (2) of FIG. 1, for cyclosporine, a subcutaneous (or “subQ” or “SQ”) regime is listed, wherein cyclosporine is injected subcutaneously for one week prior to injection of genetic material, and then every two days after, as is a cyclosporine+MMF combination (noted as “Cyc+MMF”) which has been used with success, for example, in clinical trials with adeno associated virus (“AAV”) gene therapies; the term “I.P.” or “i.p.” refers to intraperitoneal injection. Dexamethasone injected every day for one week prior to injection of genetic material, and then every two days onwards also is noted (as “Dex pre/post”), as is another dexamethasone paradigm (labelled as “Dex post”) featuring subcutaneous injection every day from the day before injection of genetic material onwards. Also listed are several rapamycin configurations, the first (“Rapa low dose”), second (“Rapa medium”), and third (“Rapa high”) requiring injection of rapamycin every day from the day before injection of genetic material onwards, with different dosing, respectively, as shown. The “Rapa/FK506/dex” paradigm listed requires injection every day from the day before injection of genetic material for thirty days, and then daily rapamycin injections every day thereafter. Finally, the “Metformin” paradigm listed in FIG. 1 requires injections of metformin every day for one week prior to injection of the genetic material, followed by continued metformin injections every two days thereafter.

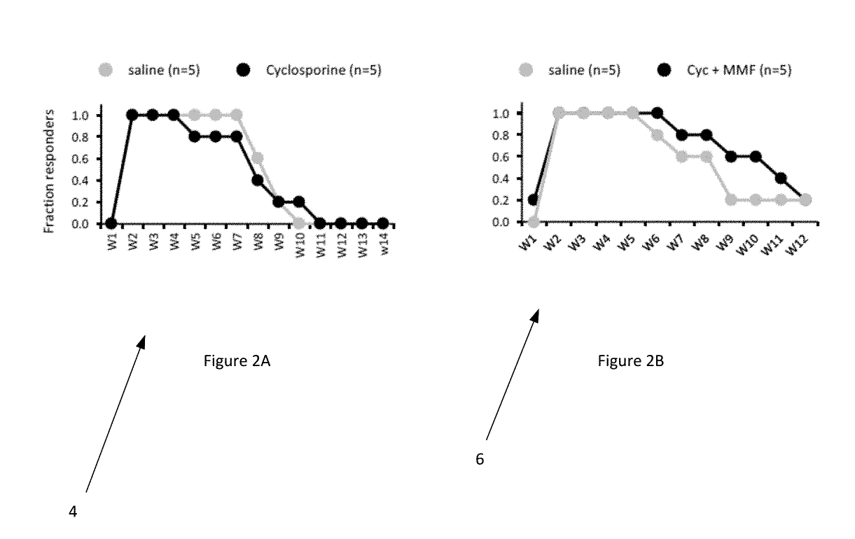

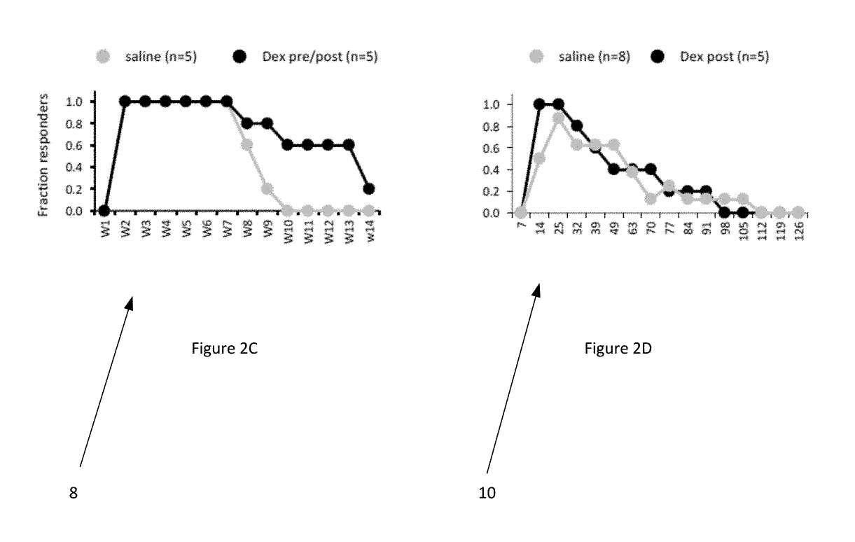

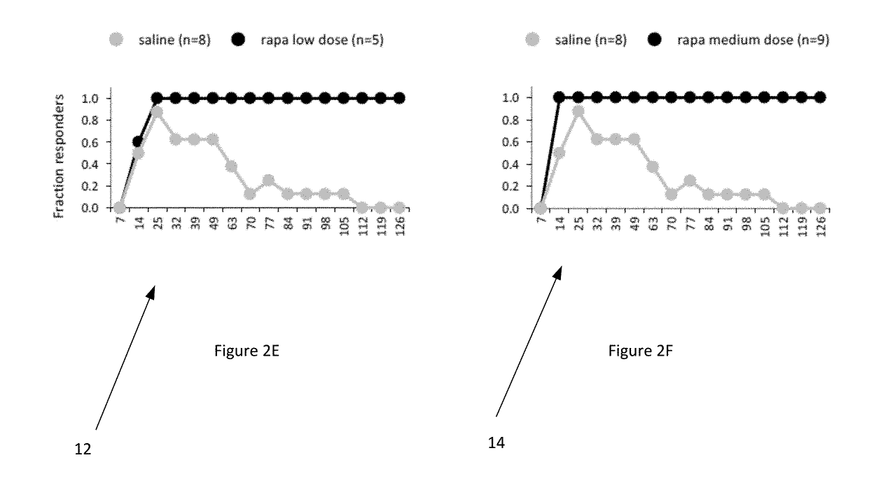

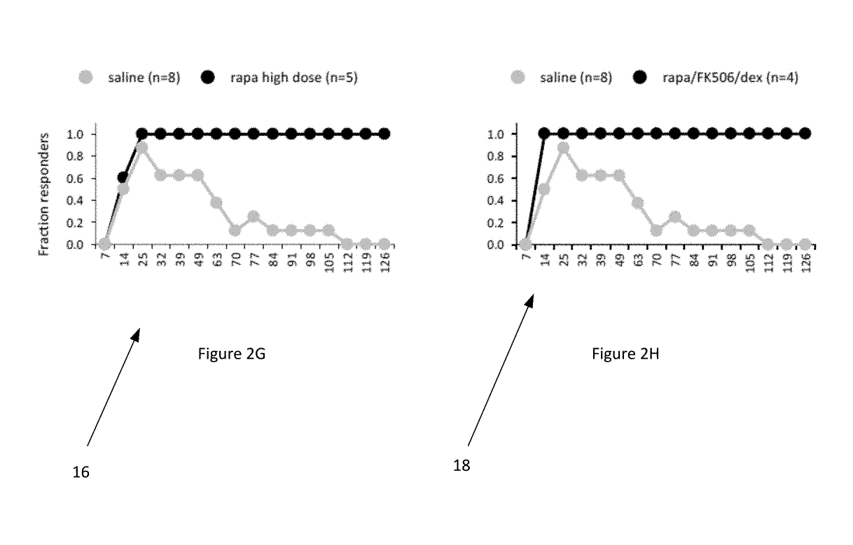

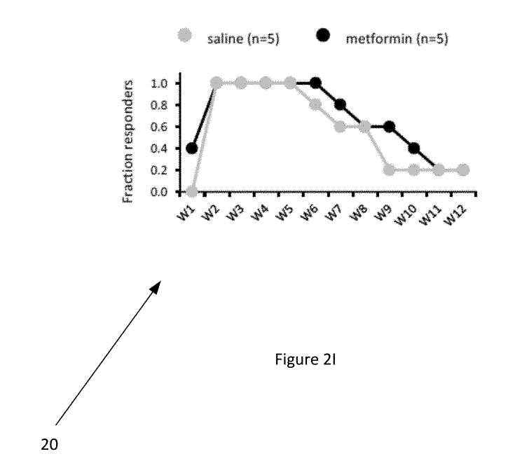

Referring to FIG. 2A (4) the vertical axis illustrates the amount of experimental animals responding as a fraction of total number of animals; the horizonal axis represents time (“W1” represents Week 1 post injection of genetic material, etc.). This chart (4) illustrates the effects of the Cyclosporine paradigm, in this experiment with the light responsive excitatory opsin protein ChR2 delivered virally via AAV and expressed in the sensory nerve of a mouse; exposure to light (at approximately 450 nm) stimulates pain-related sensory nerve tissue, with a detectable reaction in the animal; we have shown this to be a particularly efficient model. At week 1 post injection of optogenetic material, none of the animals are showing any response to light-based stimulation, presumably because it takes some time for the AAV delivery package to express light-responsive proteins. Two weeks later, however, 100% of the animals are showing light-responsiveness. The saline group represents the paradigm without immunosuppression. At eight weeks out, only about half of the animals are showing light-responsiveness, and by week 22, none are showing light-responsiveness (i.e., the animal's immune response has functioned to stop the light responsive opsin protein from working). These limited datapoints illustrate that in this experiment, the Cyclosporine paradigm, which has been suggested by literature to be effective, is not substantially effective, with all of the animals not showing light-responsiveness after about 11 weeks.

Referring to FIG. 2B, an AAV genetic material delivery injection configuration along with the cyclosporine+MMF paradigm from FIG. 1 is illustrated (6) with similar axes as in FIG. 2A (fraction of responders vs time). This cyclosporine+MMF paradigm has been shown in AAV papers with rodent models, and also in some clinical trials pertaining to human newborn diseases to be at least somewhat effective.

Referring again to FIG. 2B, a mild effect versus the saline control is shown, but is relatively minimal and is not statistically significant in our limited experiments.

Referring to FIG. 2C, data pertaining to the “Dex pre/post” configuration are illustrated in the depicted chart (8), with axes as noted above (fraction of respondents versus time after initial injection of genetic material in weeks). These data do point to a statistically significant extension of light responsiveness by an average of about three weeks, but after about fourteen weeks, all of the dexamethasone animals have lost their light-responsiveness in our limited data.

Referring to FIG. 2D, data pertaining to the “Dex post” configuration are illustrated in the depicted chart (10), with axes as noted above (fraction of respondents versus time after initial injection of genetic material in weeks). These limited results indicate that it does seem to be beneficial to have the dexamethasone loaded into the animal for a week before the injection of the genetic material to be able to get results more like those illustrated in FIG. 2C.

Referring to FIG. 2E, data pertaining to the “Raga low dose” configuration are illustrated in the depicted chart (12), with axes as noted above (fraction of respondents versus time after initial injection of genetic material, here in days). Our hypothesis that rapamycin tolerizes the animals and facilitates relatively long-lasting expression shows promise in this data, wherein at 126 days out, every single animal remains light responsive, compared to the control/saline animals, wherein at 70 days out, almost all of the animals indicate light sensitivity. Referring to FIG. 2F, data similar to that of FIG. 2E is illustrated in the chart (14) for the “Rapa medium dose” experimentation. Referring to FIG. 2G, data similar to that of FIG. 2E is illustrated in the chart (16) for the “Rapa low dose” experimentation; we note that this is a relatively low dose as compared with dosages of humans in clinical environments. Referring to FIG. 2H, data similar to that of FIG. 2E is illustrated in the chart (18) for the “Rapa/FK506/dex” experimentation.

Referring to FIG. 2I, data is charted (20) for the metformin group of experimental animals and related saline controls. Such a paradigm was shown in one academic paper to have an affect on AAV based expression and tolerization. As shown in FIG. 2I, this paradigm does not seem to have a significant impact in our experiments.

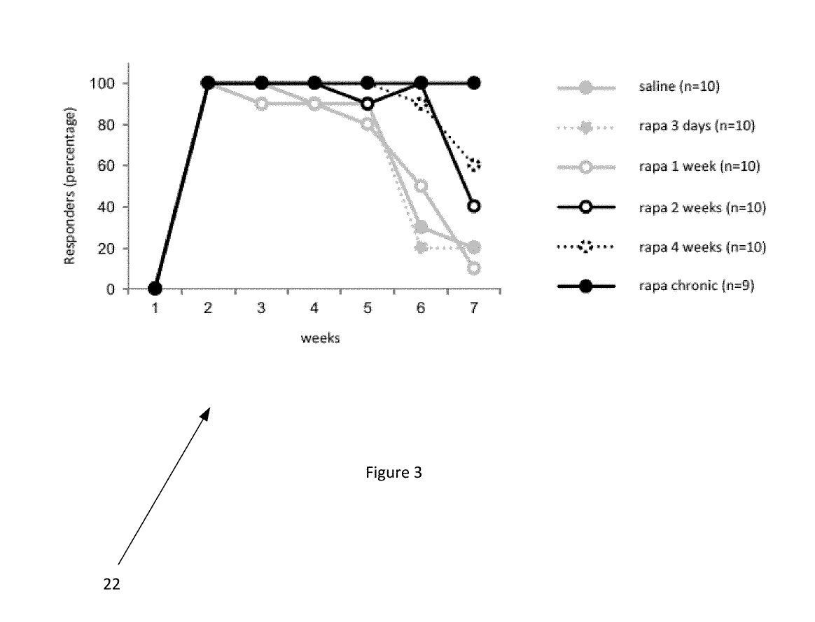

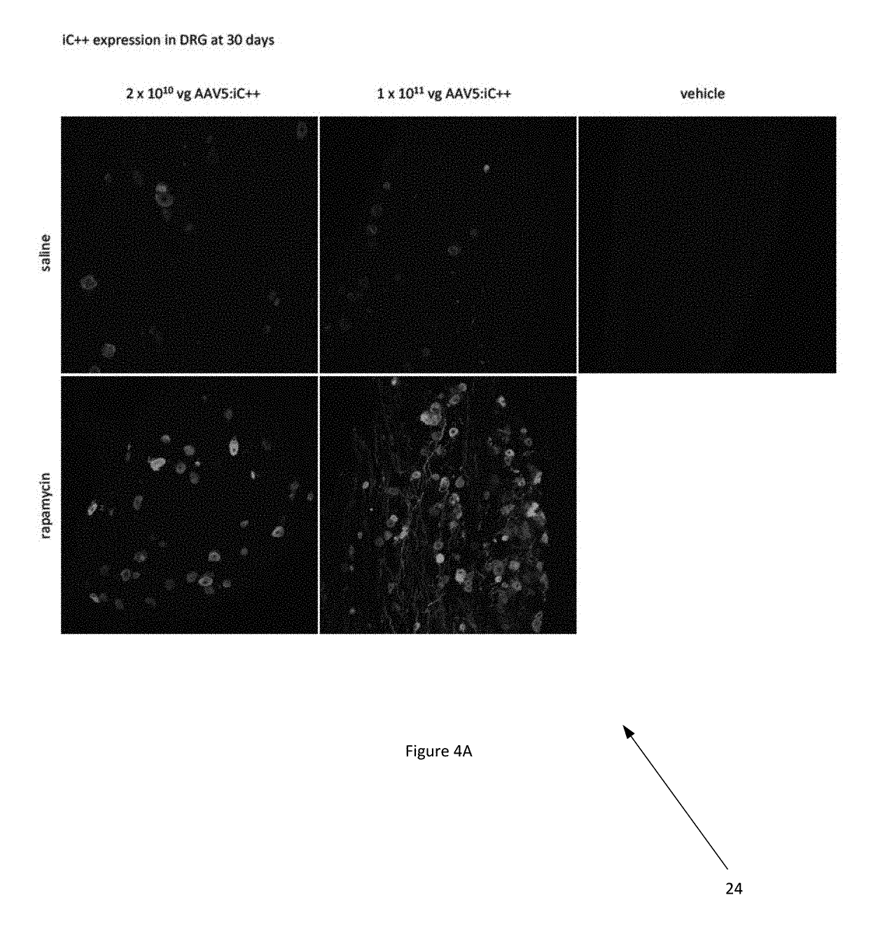

Referring to FIG. 3, experimental data pertaining to exposure time to rapamycin is charted (22) with vertical axis percentage of responders and horizontal axis time in weeks after injection of genetic material. This experiment is designed to assist in answering questions such as: low long must the rapamycin exposure last to bring about impacts on the data; must it be in the system of the animal constantly, or can it be removed at some point. The data from six groups of ten animals each is illustrated (first group saline; then rapamycin for 3 days starting at the date of genetic material injection; then 1 week, then 2 weeks, then 4 weeks, then chronic (i.e., continued the entire time after date of genetic material injection)). This experimental data indicates that the non-rapamycin saline group is dropping off in light responsiveness at about seven weeks, as would be predicted by the data discussed above. The 3-days and 1-week groups also are dropping off at about seven weeks. Two and four weeks of rapamycin appear to push the drop-off of light responsiveness out by a couple of weeks. With the experimental formulations and in accordance with this data, it appears that chronic rapamycin is preventing the drop-off on a longer term basis.

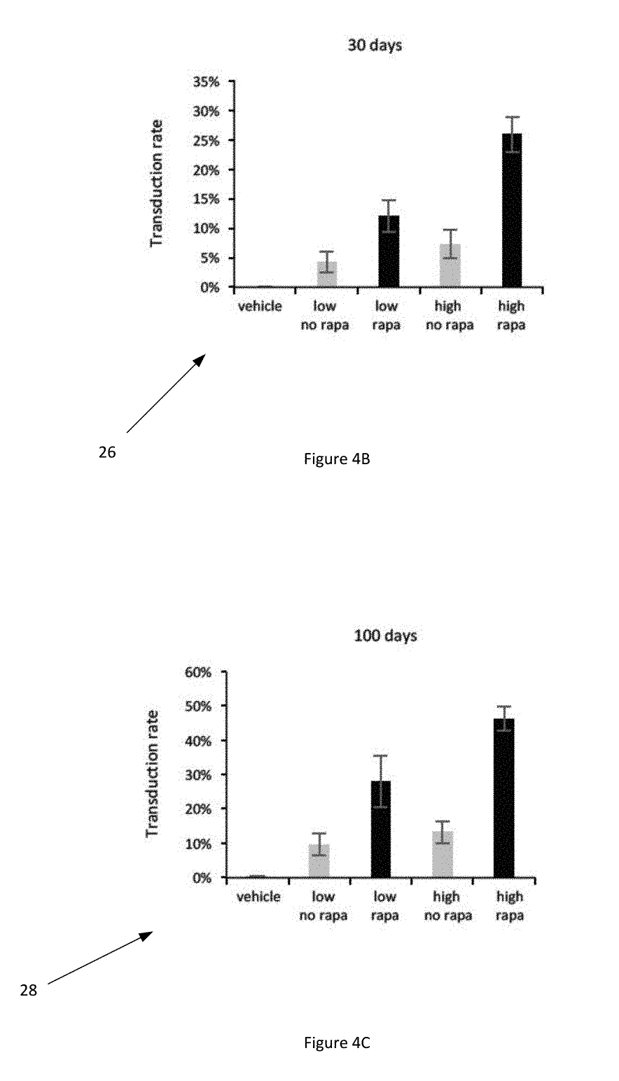

Referring to FIG. 4A, various aspects of histology (24) presentation are shown to help indicate whether the aforementioned paradigm is working only for one species and one light responsive opsin protein, or whether it seems to be generalizable across species and across other opsin proteins. The data in FIG. 4A pertain not to ChR2, as in the above experiments, but to the light responsive opsin protein IC++, injected directly into the dorsal root ganglia (“DRG”) of rats. This histology data (24) illustrates that if we sacrifice those rats 30 days after delivery of the genetic material, the saline animals (upper portion of the depicted histology data 24) have less opsin protein expression compared with the rapamycin treated animals (lower portion of the depicted histology data 24). The indication is that there is more opsin expression—indicated by more cells (also brighter appearance, indicating higher levels of expression). Thus, in this experimental data we are seeing both higher levels of expression and more cells, with the rapamycin treatment. “Vehicle” is the term utilized to indicate the experimental paradigm wherein we inject but with no virus to deliver genetic material.

Referring to FIG. 4B, transduction rate is quantified and plotted (26) for the histological data pertaining to injection without virus (vehicle), a low dose of virus and no rapamycin (low/no rapa), a low dose of virus with rapamycin (low/rapa), a high dose of virus without rapamycin (low/no rapa), and a high dose of virus with rapamycin (high/rapa). FIG. 4C illustrates similar data (28) charted but pertaining to one hundred days post injection. Just comparing “rapa” to “no rapa” for the two doses of virus, rapamycin is associated with much higher cells expressing at thirty days, as shown in FIG. 4B. Presumably this is associated with impacts of immune responses of the animals. Referring again to FIG. 4C, at one hundred days, this effect appears to be even more dramatic: for example, looing at the high rapa dose group, without rapamycin about ten percent is expressing, while with rapamycin about fifty percent is expressing; and note, again, that this is a different species, different opsin protein, relative to the experiments discussed above.

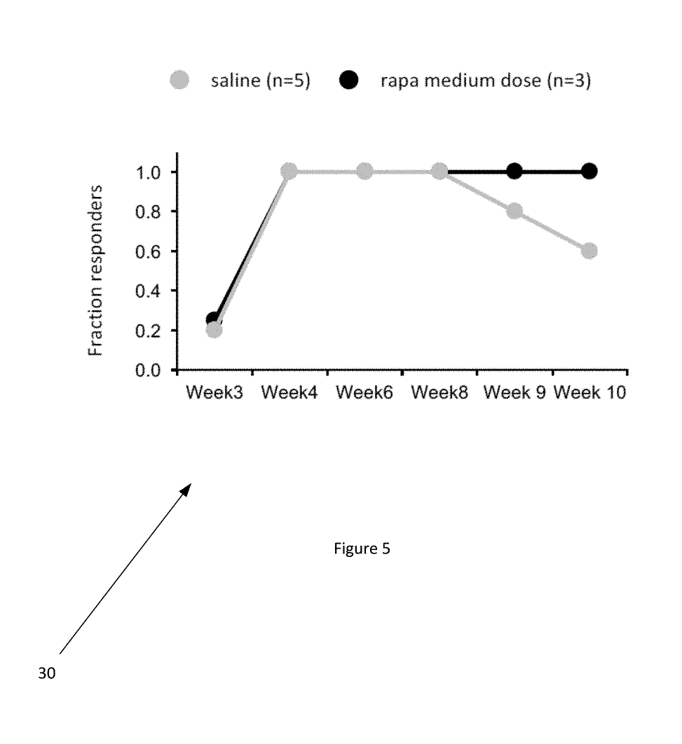

Referring to FIG. 5, data is charted (30) that pertains to whether a successful rapamycin treatment is further generalizable not just to sensory neurons, but also to motor neurons. FIG. 5 illustrates preliminary data indicating that a medium dose of rapamycin in rats can block the loss of light responsiveness function due to the immune response in a ChR2/ motor neuron model (these rats were injected in the tibialis anterior muscle with an AAV configuration to deliver ChR2 light responsive opsin protein via retrograde transport to the spinal cord motor neurons of the rats; we also injected five animals with saline and three with a medium dose of rapamycin. The data shown (30) illustrate that at ten weeks, the saline group has started to lose light responsiveness (two of the five saline animals have lost function at ten weeks); the rapamycin animals still have light responsiveness.

With regard to primate applications such as humans, a recombinant rAAV5-based gene therapy which causes target cells to express a light-sensitive protein, such as the anion channel iC++, may be utilized. The viral vector may be composed of two elements: (1) a single-stranded DNA vector genome consisting of an expression cassette for the mRNA expressing iC++along with terminal repeats that permit packaging of the DNA into the AAV5 capsid; and (2) an rAAV5-derived protein capsid within which the vector genome is packaged. The virus may be manufactured using recombinant plasmid DNA purified from bacteria to express elements for AAV5 production in human-derived cells.

The gene therapy agent may be prepared in a pharmacy that meets USP 797 guidelines governing sterile preparation of pharmaceuticals and USP 800 guidelines governing preparation and handling of hazardous drugs. A prepared solution of said agent may be transported to the clinical site as per the USP 800 guidelines governing preparation and handling of hazardous drugs.

By way of nonlimiting example, superficial pain of a lower extremity may be treated by injecting into the dorsal root ganglion (DRG) of a patient. Based upon experience with implantable spinal cord stimulators and their clinical and preclinical data demonstrating the highly focal efficacy of such isolated DRG treatment, multiple DRGs may be targeted, based upon the optimal number of DRGs necessary to address the targeted body area of pain (such as a 3 DRGs for the treatment of a distal lower extremity). In this exemplary embodiment, the patient may be sedated before the gene therapy agent infusion procedure. A 22G guide needle may be inserted toward a DRG under x-ray computed tomography (CT) guidance and the dura outside of the DRG may be penetrated. A 32G injection needle may then be passed into the DRG through the 22G guide needle. 0.2 mL of the agent may be injected over a 20 minute period, for example. The injection needle may be left in place for 5 minutes after injection and the injection and guide needle may then be removed. One DRG infusion may be completed prior to repeating the procedure at another level, for a total of, for example, 3 levels to be treated. Following administration, patients may be monitored for safety and tolerability and kept at a clinic or hospital for a minimum of 48 hours post-infusion for observation per local hospital practice

Viral doses for a patient may range from 1.0×1012 vg/mL to 4.0×1013 vg/mL.

Rapamycin (marketed using tradenames such as “sirolimus”™ or “Rapamune”™), is a macrolide antibiotic that has been used in gene therapy clinical trials to increase the stability of transgene expression. The administration of rapamycin was evaluated in the preclinical in vivo experiments to assess the effects on long-term expression of the iC++ transgene. Indeed, daily administration of rapamycin reduced inflammation and increased iC++ expression following intraganglionic administration of iC++ using the neuropathic pain model in rats. When delivered to DRG neurons mediating pain, there is a quantitative reduction in hyperpathic pain in pre-clinical models of neuropathic pain. Although the above description focuses on iC++, other opsins have shown similar success and are also considered within the scope of the present invention.

Patients receiving this treatment may take a 2 mg dose of Rapamycin for a 3 month duration, except on the first day for which patients may take a loading dose of 6 mg. Dosages may also be increased by as much as 3×. Similar compounds for maintenance immunosuppression may include all immunosuppressive medications given before, during or after infusion with the intention of long-term maintain them long-term, such as Prednisone, Cyclosporine, Tacrolimus, Mycophenolate Mofetil, Azathioprine and Rapamycin; as has been described. In addition, maintenance immunosuppression does not include any immunosuppressive medications given to treat rejection episodes, or for induction. Patients may also undergo a regime of induction immunosuppression. This approach may include all medications given immediately after infusion in intensified doses for the purpose of preventing acute rejection. Although the drugs may often be continued for the first 30 days after infusion, they may also be used long-term for immunosuppressive maintenance. Associated medications can include Methylprednisolone, Atgam™, Thymoglobulin™, OKT3, Basiliximab and Daclizumab. These compounds may be used singly, in combination, and with the addition of steroids, such as corticosteroids.

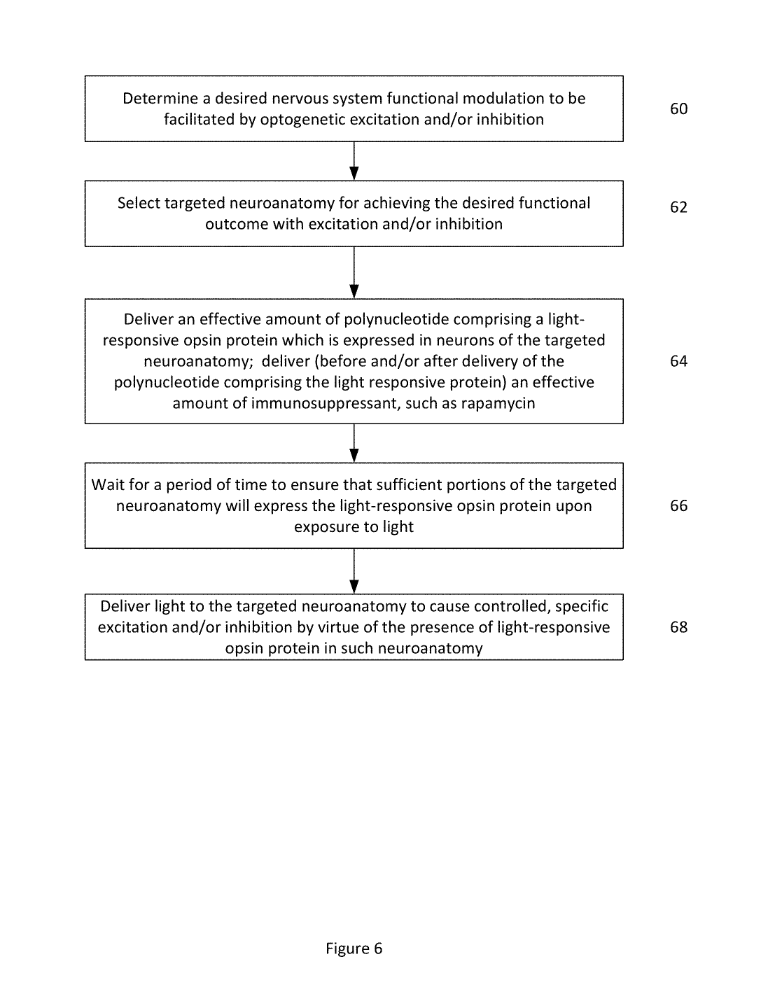

Referring to FIG. 6, in a typical method configuration for nonlimiting exemplary purposes, a desired nervous system functional modulation to be facilitated by optogenetic excitation and/or inhibition may be determined (60); neuroanatomy may be selected for achieving the desired functional outcome with excitation and/or inhibition (62); an effective amount of polynucleotide comprising a light-responsive opsin protein which is expressed in neurons of the targeted neuroanatomy may be delivered, as well as (before and/or after delivery of the polynucleotide comprising the light responsive protein) an effective amount of immunosuppressant, such as rapamycin (64); after a period of time sufficient to ensure that portions of the targeted neuroanatomy will express the light-responsive opsin protein upon exposure to light (66), light may be delivered to the targeted neuroanatomy to cause controlled, specific excitation and/or inhibition by virtue of the presence of light-responsive opsin protein in such neuroanatomy (68).

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed:

1. A method for treating the nervous system of a patient, comprising:
a. determining a desired nervous system functional modulation to be facilitated by optogenetic intervention;
b. selecting targeted neuroanatomy for achieving the desired functional outcome with optogenetic intervention; and
c. delivering an effective amount of polynucleotide comprising a light-responsive opsin protein which is expressed in neurons of the targeted neuroanatomy, and delivering an effective amount of immunosuppressant.

2. The method of claim 1, further comprising waiting for a period of time to ensure that sufficient portions of the targeted neuroanatomy will express the light-responsive opsin protein upon exposure to light.

3. The method of claim 1, further comprising delivering light to the targeted neuroanatomy to cause controlled, specific excitation and/or inhibition by virtue of the presence of light-responsive opsin protein in such neuroanatomy.

4. The method of claim 1, wherein determining a desired nervous system functional modulation to be facilitated by optogenetic intervention comprises determining a desired nervous system functional modulation to be facilitated by optogenetic excitation.

5. The method of claim 1, wherein determining a desired nervous system functional modulation to be facilitated by optogenetic intervention comprises determining a desired nervous system functional modulation to be facilitated by optogenetic inhibition.

6. The method of claim 1, wherein determining a desired nervous system functional modulation to be facilitated by optogenetic intervention comprises determining a desired nervous system functional modulation to be facilitated by optogenetic excitation and inhibition.

7. The method of claim 1, wherein selecting targeted neuroanatomy for achieving the desired functional outcome with optogenetic intervention comprises selecting targeted neuroanatomy for achieving the desired functional outcome with optogenetic excitation.

8. The method of claim 1, wherein selecting targeted neuroanatomy for achieving the desired functional outcome with optogenetic intervention comprises selecting targeted neuroanatomy for achieving the desired functional outcome with optogenetic inhibition.

9. The method of claim 1, wherein selecting targeted neuroanatomy for achieving the desired functional outcome with optogenetic intervention comprises selecting targeted neuroanatomy for achieving the desired functional outcome with optogenetic excitation and inhibition.

10. The method of claim 1, wherein the effective amount of immunosuppressant is delivered before delivering the effective amount of polynucleotide comprising a light-responsive opsin protein which is expressed in neurons of the targeted neuroanatomy.

11. The method of claim 1, wherein the effective amount of immunosuppressant is delivered concomitantly with delivering the effective amount of polynucleotide comprising a light-responsive opsin protein which is expressed in neurons of the targeted neuroanatomy.

12. The method of claim 1, wherein the effective amount of immunosuppressant is delivered before and also concomitantly with delivering the effective amount of polynucleotide comprising a light-responsive opsin protein which is expressed in neurons of the targeted neuroanatomy.

13. The method of claim 1, wherein the immunosuppressant is rapamycin.
